(19) United States
(12) Patent Application Publication
(10) Pub. No.: US 2020/0166514 A1
(43) Pub. Date: May 28, 2020

(54) METHODS AND COMPOSITIONS FOR QUANTIFYING EXOSOMES

(71) Applicant: Aethlon Medical, Inc., San Diego, CA (US)

(72) Inventors: Richard H. Tullis, Encinitas, CA (US); Paul Duffin, San Diego, CA (US)

(21) Appl. No.: 16/459,220

(22) Filed: Jul. 1, 2019

Related U.S. Application Data
(63) Continuation of application No. 13/808,561, filed on Aug. 14, 2013, now abandoned.
(60) Provisional application No. 61/362,129, filed on Jul. 7, 2010.

(51) Int. Cl. G01N 33/574; G01N 33/569

(52) U.S. Cl. CPC G01N 33/57492; G01N 33/57449; G01N 33/5743; G01N 2333/4724; G01N 33/57446; G01N 33/56966; G01N 33/5695

(57) ABSTRACT

Embodiments of the present invention relate to methods, compositions and kits for quantifying exosomes. In particular, methods, composition and kits that utilize lectins to quantify exosomes are provided.

### RELATED APPLICATIONS

This application is a continuation of U.S. application Ser. No. 13/808,561, filed on Aug. 14, 2013, which is a national phase application of PCT/US2011/043265, filed on Jul. 7, 2011, which designated the United States and was published in English and, which claims priority to U.S. Provisional Application No. 61/362,129 filed Jul. 7, 2010 entitled “METHODS AND MATERIALS FOR DETECTION AND QUANTIFICATION OF EXOSOMES” the contents of each of which is hereby incorporated by reference in its entirety.

### FIELD OF THE INVENTION

Embodiments of the present invention relate to methods, compositions and kits for quantifying exosomes. In particular, methods, composition and kits that utilize lectins to quantify exosomes are provided.

### BACKGROUND OF THE INVENTION

Exosomes include small vesicles released by mammalian cells for a number of purposes including immunomodulation. Depending on the conditions, exosomes can either be immunostimulatory or immunosuppressive. During pregnancy, exosomes inhibit the production of certain T-cells thereby protecting the fetus (Taylor, D. D., et al., Pregnancy-associated exosomes and their modulation of T cell signaling. J Immunol, 2006. 176(3): 1534-42). In the case of certain bacterial infections, exosomes derived from infected cells express antigenic fragments of the bacterium to stimulate the immune system against the pathogen (Bhatnagar, S., et al., Exosomes released from macrophages infected with intracellular pathogens stimulate a proinflammatory response in vitro and in vivo. Blood, 2007. 110(9): 3234-44). It has been postulated that cancers utilize the immunomodulatory properties of exosomes in order to evade the immune system (Taylor D. D. et al., (2005) Tumor-derived exosomes and their role in cancer associated T-cell signaling defects. Brit. J. of Cancer 92:305-311).

While there is increasing evidence of the importance of exosomes in the progression and prognosis of cancers and infectious diseases both as a means of treatment and as a diagnostic, there is not currently an assay that reliably detects exosomes from a number of cell types. Exosomes are currently characterized and purified by methods such as size chromatography and general protein assay. Exosomes may also be purified using antibodies specific to particular exosome epitopes (See, e.g., U.S. Pat. App. No. 20090220944, incorporated herein by reference in its entirety). However, methods using such antibodies are limited to exosomes that display particular antigens only. In view of the increasing importance of exosomes in the diagnosis and prognosis of certain diseases, there is an need for simple and efficient methods to quantify exosomes that is generally applicable to exosomes from a number of cell types.

### SUMMARY OF INVENTION

Embodiments of the present invention relate to methods, compositions and kits for quantifying exosomes. In particular, methods, composition and kits that utilize lectins to quantify exosomes are provided. Some embodiments include methods for quantifying exosomes in a biological sample. Some such embodiments include contacting the sample with lectin immobilized on a substrate; contacting exosomes bound to the lectin with a detectable exosome-binding agent; and measuring a signal from the bound detectable exosome-binding agent, thereby quantifying the bound exosomes. Some embodiments include comparing the signal from the bound detectable binding agent to a signal on a standard curve. In some embodiments, the standard curve comprises a mannan or mannan bead standard curve. In some embodiments, the exosomes are derived from a cell selected from the group consisting of an ovarian cancer cell, a melanoma cell, a colon cancer cell, and a tuberculosis infected cell. In some embodiments, the method provides a sensitivity of detecting exosomes in the sample that is at least about 1×10⁹ exosomes/ml. In some embodiments, the method provides a sensitivity of detecting exosomes in the sample of at least about 1×10⁸ exosomes/ml. In some embodiments, the method provides a sensitivity of detecting exosomes in the sample of at least about 1×10⁷ exosomes/ml. In some embodiments, the method provides a sensitivity of detecting exosomes in the sample of at least the equivalent of about 1000 pg mannan/ml. In some embodiments, the method provides a sensitivity of detecting exosomes in the sample of at least the equivalent of about 500 pg mannan/ml. In some embodiments, the method provides a sensitivity of detecting exosomes in the sample of at least the equivalent of about 100 pg mannan/ml. In some embodiments, the sample comprises an exosome having a diameter of about 10 nm to about 800 nm. In some embodiments, the sample comprises an exosome having a diameter of about 30 nm to about 200 nm.

In some embodiments, the sample comprises exosomes isolated from a fluid by a method selected from the group consisting of size exclusion chromatography, density gradient centrifugation, differential centrifugation, nanomembrane ultrafiltration, immunoabsorbent capture, affinity purification, microfluidic separation, and a combination thereof.

In some embodiments, at least one of the lectin immobilized on the substrate and the detectable binding agent are capable of binding to exosomes derived from a plurality of cell types. In some embodiments, the detectable exosome-binding agent comprises a lectin, an antibody, or an antibody fragment. In some embodiments, the lectin immobilized on the substrate or the detectable exosome-binding agent is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A. In some embodiments, the detectable exosome-binding agent comprises a detectable label selected from the group consisting of horse radish peroxidase, and fluorescein.

In some embodiments, the biological sample is selected from the group consisting of peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, and ascites fluid. In some embodiments, the biological sample is mammalian. In some embodiments, the biological sample is human.

In some embodiments, the substrate comprises a material selected from the group consisting of sepharose, latex, glass, polystyrene, polyvinyl, nitrocellulose and silicon. In some embodiments, the substrate comprises a multiwell plate.

Some embodiments include kits for quantifying exosomes in a biological sample. Some such kits include a substrate with lectin immobilized thereon; and a detectable exosome-binding agent. In some embodiments, at least one of the lectin immobilized on a substrate and the detectable agent are capable of binding to exosomes derived from a plurality of cell types. In some embodiments, the detectable agent is capable of binding to exosomes derived from a plurality of cell types. In some embodiments, the kit provides a sensitivity of detecting exosomes in the sample that is at least about 1×10⁹ exosomes/ml. In some embodiments, the kit provides a sensitivity of detecting exosomes in the sample that is at least about 1×10⁸ exosomes/ml. In some embodiments, the kit provides a sensitivity of detecting exosomes in the sample that is at least about 1×10⁹ exosomes/ml. In some embodiments, the kit provides a sensitivity of detecting exosomes in the sample that is at least the equivalent of about 1000 μg mannan/ml. In some embodiments, the kit provides a sensitivity of detecting exosomes in the sample that is at least the equivalent of about 500 μg mannan/ml. In some embodiments, the kit provides a sensitivity of detecting exosomes in the sample that is at least the equivalent of about 100 μg mannan/ml.

In some embodiments, the detectable exosome-binding agent comprises a lectin, an antibody, or an antibody fragment. In some embodiments, the lectin immobilized on the substrate or the detectable exosome-binding agent is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A. In some embodiments, the detectable exosome-binding agent comprises a detectable label selected from the group consisting of an enzyme, a chemiluminescent agent, a fluorescent agent, and an isotope.

In some embodiments, the substrate comprises a material selected from the group consisting of sepharose, latex, glass, polystyrene, polyvinyl, nitrocellulose and silicon. In some embodiments, the substrate comprises a multiwell plate.

### BRIEF DESCRIPTION OF DRAWINGS

FIG. 1 is a plot of absorbance at 450 nm versus concentration of mannan measured by an embodiment of an assay of the present invention.

FIG. 2 is a plot of absorbance at 450 nm versus concentration of mannan coated latex beads measured by an embodiment of an assay of the present invention.

FIG. 3 depicts a mannan standard curve and a purified exosome preparation measured by an embodiment of an assay of the present invention.

FIG. 4 depicts a dilution of an exosome sample measured by an embodiment of an assay of the present invention.

FIGS. 5A and 5B depict a mannan (FIG. 5A) and mannan coated latex bead (FIG. 5B) optimization experiment where known quantities of mannan and mannan beads were incubated in 96 well plate wells that were coated with different amounts of GNA (10 μg/ml, 5 μg/ml, 2.5 μg/ml, and 1.25 μg/ml).

FIG. 6 depicts a mannan bead standard curve measured by an embodiment of an assay of the present invention.

FIG. 7 depicts a graph of quantities of exosomes present in a sucrose-gradient purified sample or a GNA column purified sample, relative to a mannan standard curve measured by an embodiment of an assay of the present invention.

FIG. 8 depicts a graph of ovarian cancer exosomes detected using an anti-PLAP antibody in an embodiment of an assay of the present invention.

### DETAILED DESCRIPTION

Embodiments of the present invention relate to methods, compositions and kits for quantifying exosomes. In particular, methods, composition and kits that utilize lectins to quantify exosomes are provided. Lectins such as concanavalin A and Galanthus Nivalis lectin (GNA) bind specifically to cancer cells indicating that these cells contain sugar moieties uncommon to healthy cells. Cancer specific exosomes secreted from these cells have the same moieties, and are therefore be available to lectin capture and detection.

Assays such as the enzyme linked immunosorbent assay (ELISA) is a powerful tool that has been used for the past 4 decades to detect proteins in homogenous and heterogenous samples, generally by adsorbing an antigen specific antibody to a solid surface such as a 96 well plastic plate, incubating the antigen with the adsorbed antibody, and detecting the antigen with a secondary antibody labeled with various chemicals that react to give color, fluorescence or other means of detection.

However, the use of ELISA for the detection of exosomes requires an antibody which is specific for a surface antigen on the exosome which is not present on normal cells. The development of antibodies for use with an ELISA assay can be difficult and tedious. Therefore, there is a need for methods and compositions that specifically and reliably capture and/or detect exosomes, preferably in a quantitative manner.

Beyond the potential therapeutic benefits of eliminating immunosuppressive exosomes from circulation, researchers recognize that exosomes represent an important diagnostic target to determine progression and prognosis of both cancers and infectious disease conditions. However, the availability of functional assays that specifically detect exosomes is limited. At present, exosomes are generally characterized and purified by size chromatography and general protein assay, not by chemical structures specific to the exosome. Some embodiments of the present technology include Enzyme Linked Lectin Specific Assays (ELLSA) designed to bind specifically to carbohydrate structures common to exosomes, but not to healthy human cellular components. In some such embodiments, each ELLSA plate allows for up to 96 exosome detection tests. Further analysis of the captured exosomes is possible through detection molecules such as antibodies linked to a specific biomarker on the exosome.

The Hemopurifier® is a medical device that selectively targets the removal of infectious viruses and immunosuppressive proteins from the entire circulatory system (See, e.g., U.S. Pat. No. 7,226,429, incorporated by reference herein in its entirety). It has been discovered that devices such as the Hemopurifier® capture tumor-secreted exosomes that suppress the immune system of those afflicted with cancer (See e.g., U.S. Pat. App. No. 20090304677, incorporated by reference herein in its entirety). Prior to this discovery, a therapeutic strategy to directly inhibit or reverse the immunosuppressive destruction caused by exosomes did not exist in cancer care. By eliminating this mechanism, the Hemopurifier® can fill an unmet clinical need and provide the benefit of an immune-based therapy without adding drug toxicity or interaction risks to established and emerging treatment strategies

In some embodiments, the Hemopurifier® is a selective filtration device containing affinity agents that tightly bind to high-mannose structures unique to the surface of exosomes produced by cancer and glycoproteins residing on the envelope of viruses. Agents can include some biomarkers (See, e.g., Int. Pat. Pub. No. WO 2010/065765, incorporated herein by reference in its entirety). These agents are immobilized around approximately 2800 porous hollow fibers that run the interior length of the device. The resulting design enhances the ability to separate both exosome and viral targets away from blood cells so they can then be selectively and permanently removed from the circulatory system. In some applications, blood circulation is established into the Hemopurifier® via a catheter or other blood access device. Once blood flow has been established, treatment benefit is immediate as the entire circulatory system can pass through the Hemopurifier®, in some embodiments, in as little as 15 minutes. However, there remains a need to measure the concentration of exosomes in patient samples to gauge the effectiveness of the Hemopurifier® treatment. Embodiments of the present invention satisfy this need by providing methods, compositions and kits for quantifying a broad spectrum of exosomes in samples.

### Biological Samples

Some embodiments of the methods and compositions provided herein include a biological sample. Biological samples include, for example, cell culture media, and samples obtained from a subject. As used herein, the term “subject” includes an animal, a mammal, and a human. A sample obtained from a subject can include any tissue or fluid from the subject that may contain exosomes. Examples of biological samples obtained from a subject that may contain exosomes include peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation which may be of fetal or maternal origin. The biological sample may also be a tissue sample or biopsy, from which exosomes may be obtained. For example, if the sample is a solid sample, cells from the sample can be cultured and exosome product induced. In some embodiments, the sample is ascites fluid from a subject, e.g., ascites fluid from a human subject with ovarian cancer; cell culture media supernatant from a human primary melanoma cell line; cell culture media supernatant from a human primary colon cancer cell line; or murine macrophage, e.g., murine macrophage infected with tuberculosis.

### Exosomes

Some embodiments of the methods and compositions provided herein include a sample comprising exosomes. Generally, exosomes are small vesicles that are released into the extracellular environment from a variety of different cells, for example, cells that originate from, or are derived from, the ectoderm, endoderm, or mesoderm including any such cells that have undergone genetic, environmental, and/or any other variations or alterations. An exosome is typically created intracellularly when a segment of the cell membrane spontaneously invaginates and is ultimately exocytosed (see e.g., Keller et al. (2006), Immunol. Lett. 107: 102-8, incorporated by reference herein in its entirety). In some embodiments, the exosomes have a diameter of greater than about 10 nm, 20 nm, or 30 nm; a diameter that is, or is about, 30-1000 nm, 30-800 nm, 30-200 nm, or 30-100 nm. In some embodiments, exosomes have a diameter of less than, or less than about, 10,000 nm, 1000 nm, 800 nm, 500 nm, 200 nm, 100 nm or 50 nm. Exosomes may also be referred to as microvesicles, nanovesicles, vesicles, dexosomes, bleb, blebby, prostasomes, microparticles, intralumenal vesicles, endosomal-like vesicles or exocytosed vehicles. Exosomes can also include any shed membrane bound particle that is derived from either the plasma membrane or an internal membrane. Exosomes can also include cell-derived structures bounded by a lipid bilayer membrane arising from both herniated evagination separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen including tumor-derived microRNAs or intracellular proteins. Exosomes can also include membrane fragments.

In some embodiments, the exosomes are cancer exosomes. Cancer exosomes include exosomes from cancer cells and/or tumor cells (primary or cell culture) from cancers such as breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer includes CRC Dukes B or Dukes C-D. The hematological malignancy includes B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma. Cancer exosomes may also be derives from a premalignant condition, for example, but not limited to, Barrett's Esophagus. In some embodiments, cancer exosomes include exosomes from ovarian cancer, colon cancer, and melanoma.

### Isolation of Exosomes

Some embodiments of the methods and compositions provided herein include isolating exosomes from a sample. As used herein, the term “isolating” refers to increasing the concentration or density of exosomes in a sample and, or, removing non-exosome substances (e.g., proteins, cells) from a sample. Methods of isolating exosomes are well known in the art. Examples of methods useful to isolate exosomes include but are not limited to size exclusion chromatography, density gradient centrifugation, differential centrifugation, nanomembrane ultrafiltration, immunoabsorbent capture, affinity purification, microfluidic separation, or combinations thereof. Size exclusion chromatography, such as gel permeation columns, centrifugation or density gradient centrifugation, and filtration methods can be used. For example, exosomes can be isolated by differential centrifugation, anion exchange and/or gel permeation chromatography (e.g., U.S. Pat. Nos. 6,899,863 and 6,812,023, each of which is incorporated by reference herein in its entirety), sucrose density gradients, organelle electrophoresis (e.g., U.S. Pat. No. 7,198,923, which is incorporated by reference herein in its entirety), magnetic activated cell sorting (MACS), or with a nanomembrane ultrafiltration concentrator. Various combinations of isolation or concentration methods can be used.

### Substrates

Some embodiments of the methods and compositions provided herein include a substrate. In some embodiments the substrate can comprise a surface. Substrates can include but are not limited to, for example, plates, beads, and fibers. In some embodiments, a substrate comprises a multiwell plate, such as a standard 96-well plate. A substrate can comprise any suitable materials. Examples of suitable materials include but are not limited to sepharose, latex, glass, polystyrene, polyvinyl, nitrocellulose and silicon.

### Exosome-Binding Agents

Some embodiments of the methods and compositions provided herein include an exosome-binding agent. In some embodiments, an exosome-binding agent can be associated with a substrate, for example, the exosome-binding agent can be immobilized on a substrate, in some embodiments the substrate comprises a surface. In some embodiments, an exosome-binding agent is capable of binding exosomes derived from a plurality of different sources, such as different cell types, including different cancer cell types. Examples of exosome-binding agents include but are not limited to lectins, antibodies and exosome-binding fragments of antibodies. Examples of lectins useful with some embodiments provided herein include mannose-binding lectins. Examples of lectins useful with some embodiments provided herein include Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A. In some embodiments, the lectin is GNA, NPA, SNA, or MAL. In particular embodiments, the lectin is GNA.

Antibodies and exosome-binding fragments of antibodies useful in some embodiments provided herein include those that can bind particular markers associated with exosomes. Examples of biomarkers associated with exosomes include CD63, EpCam, CD81, CD9, PCSA, PSMA, B7H3, TNFR, MFG-E8, Rab, STEAP, 5T4, or CD59 (see e.g., Escrevetne C. et al., (2011) “Interaction and uptake of exosomes by ovarian cancer cells” BMC Cancer 11:108; and U.S. Pat. App. No. 20110151460, each of which is incorporated by reference herein in its entirety).

### Detectable Moieties

Some embodiments of the methods and compositions provided herein include an exosome-binding agent or a secondary binding agent comprising a detectable moiety. Examples of detectable moieties include but are not limited to enzymes, such as horseradish peroxidase (HRP), alkaline phosphatase (AP), (3-galactosidase and urease. A horseradish-peroxidase detection system can be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which can be used with the chromogenic substrate p-nitrophenyl phosphate to yield a soluble product readily detectable at 405 nm. Similarly, a β-galactosidase detection system can be used with the chromogenic substrate o-nitrophenyl-β-D-galactopyranoside (ONPG) to yield a soluble product detectable at 410 nm, or a urease detection system can be used with a substrate such as urea-bromocresol purple (Sigma Immunochemicals, St. Louis, Mo.).

More examples of detectable moieties include but are not limited to chemiluminescent labels. Methods of detecting chemiluminescent labels are known in the art. Fluorescent detection also can be useful in certain methods provided herein. Useful fluorochromes include but are not limited to, DAPI, fluorescein, Hoechst 33258, R-phycocyanin, B-phycoerythrin, R-phycoerythrin, rhodamine, Texas red and lissamine. Fluorescein or rhodamine labeled antibodies, or fluorescein- or rhodamine-labeled secondary antibodies can be useful with embodiments provided herein. An example of a secondary antibody includes an anti-GNA antibody. Isotopes can also be useful in certain methods provided herein. Such moieties and assays are well known in the art.

A signal from a detectable moiety can be analyzed, for example, using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of a biomarker can be performed using a spectrophotometer such as an EMAX Microplate Reader (Molecular Devices; Menlo Park, Calif.) in accordance with the manufacturer's instructions. The assays of the invention can be automated or performed robotically, if desired, and that the signal from multiple samples can be detected simultaneously.

### Methods for Quantifying Exosomes

Embodiments of the methods provided herein include quantifying exosomes in a biological sample. In a particular embodiment, exosomes bound to a lectin immobilized on a substrate are detected and/or quantified using a detectable exosome-binding agent comprising a lectin. In another embodiment, exosomes bound to a lectin immobilized on a substrate are detected and/or quantified using a detectable exosome-binding agent comprising an exosome-binding antibody, or fragment thereof. In some embodiments, the detectable exosome binding agent is detected using an secondary binding agent which is labeled with a detectable moiety. For example, a secondary antibody which recognizes the detectable exosome-binding agent.

In an exemplary embodiment, a biological sample comprising exosomes is contacted with a substrate having a surface with lectin immobilized thereon. The biological sample is incubated with the immobilized lectin, and exosomes bind to the immobilized lectin. Unbound biological sample is washed from the surface. The bound exosomes are detected and measured using a detectable exosome-binding agent, e.g., an agent comprising a labeled lectin, or a labeled antibody or labeled fragment thereof. Detecting and measuring includes contacting the bound exosomes with the detectable exosome-binding agent; incubating the agent with the bound exosomes; and washing unbound agent from the exosomes bound to the lectin immobilized on the substrate. Signal from the bound detectable exosome-binding agent can be measured (optionally by contacting the exosome-binding agent with a secondary labeled binding agent) and the level of signal can be used to determine the quantity of exosomes bound to the lectin immobilized on the substrate. Methods to determine the quantity of bound exosomes using a measured signal can include comparing the level of the signal to a reference, such as a standard curve. An example standard curve includes a curve prepared using a control with various quantities of a lectin-binding compound, such as mannan or mannan coated beads. In some embodiments, the method optionally includes isolating exosomes in a sample before contacting the sample with lectin immobilized to a substrate. Methods for isolating exosomes are well known in the art and examples are also provided herein.

Exosome binding agent bound to an exosome can be detected and measured by a variety of methods. Preferably, the exosome-binding agent comprises a detectable moiety. The detectable moiety can be detected and measured using methods well known in the art; examples of such methods are provided herein. In some embodiments, the signal from the detectable moiety can be compared to a signal of a reference, for example, a standard curve. Using such well known methods, the relative quantity of bound detectable moiety can be determined. Accordingly, the relative quantity of exosomes bound by the exosome-binding moiety comprising the detectable moiety can be determined.

In some embodiments of the methods provided herein, the sensitivity of the method of detecting exosomes in a sample is, is about, is at least, or is at least about, 1×10¹⁰ exosomes/ml, 1×10⁹ exosomes/ml, 1×10⁸ exosomes/ml, 1×10⁷ exosomes/ml, or 1×10⁶ exosomes/ml, or a range defined by any two of the preceding values. In some embodiments of the methods provided herein, the sensitivity of detecting exosomes is relative to a reference, e.g., mannan-coated beads. In such embodiments, the sensitivity of detecting exosomes in a sample is equivalent to, is equivalent to about, is equivalent to at least, or is equivalent to at least about, 1×10¹⁰ beads/ml, 1×10⁹ beads/ml, 1×10⁸ beads/ml, 1×10⁷ beads/ml, or 1×10⁶ beads/ml, or a range defined by any two of the preceding values. In some embodiments, the sensitivity of detecting exosomes in the sample is, is about, is at least, or is at least about, the equivalent of 1000 ng mannan/ml, 500 ng mannan/ml, 100 ng mannan/ml, 50 ng mannan/ml, 1000 μg mannan/ml, 500 μg mannan/ml, 100 μg mannan/ml, or 50 μg mannan/ml, or a range defined by any two of the preceding values.

### Compositions and Kits for Quantifying Exosomes

Embodiments provided herein include compositions and kits for quantifying exosomes in a biological sample. In some embodiments, a composition or kit comprises a substrate with lectin immobilized thereon and a detectable exosome-binding agent. In some embodiments, the detectable agent is capable of binding to exosomes derived from a plurality of cell types. Preferably, the detectable exosome-binding agent binds specifically or preferentially to cancerous exosomes.

In one embodiment, a kit for quantifying exosomes in a biological sample comprises a substrate with lectin immobilized thereon, e.g., a 96-well plate, and a detectable exosome-binding agent, e.g., a labeled lectin. In some embodiments, the label comprises horse radish peroxidase. The detectable exosome-binding agent can be prepared to be stable with an extended shelf-life, e.g., freeze-dried and/or frozen. A kit can further comprise reagents such as diluents, wash solutions, and/or substrates for detecting the detectable exosome-binding agent in an assay. An example substrate includes tetramethylbenzadine, a substrate useful with a detectable label such as horse radish peroxidase. In some embodiments, the detectable exosome-binding agent is not labeled, and a secondary binding agent which is labeled and which binds the detectable exosome-binding agent is included.

In some embodiments of the kits provided herein, the sensitivity of the kit for detecting exosomes in a sample is, is about, is at least, or is at least about, 1×10¹⁰ exosomes/ml, 1×10⁹ exosomes/ml, 1×10⁸ exosomes/ml, 1×10⁷ exosomes/ml, or 1×10⁶ exosomes/ml, or a range defined by any two of the preceding values. In some embodiments of the methods provided herein, the kit provides a sensitivity of detecting exosomes in a sample as compared to mannan coated beads that is equivalent to, is equivalent to about, is equivalent to at least, or is equivalent to at least about, 1×10¹⁰ beads/ml, 1×10⁹ beads/ml, 1×10⁸ beads/ml, 1×10⁷ beads/ml, or 1×10⁶ beads/ml, or a range defined by any two of the preceding values. In some embodiments, the sensitivity of the kit for detecting exosomes in the sample is, is about, is at least, or is at least about, the equivalent of 1000 ng mannan/ml, 500 ng mannan/ml, 100 ng mannan/ml, 50 ng mannan/ml, 1000 μg mannan/ml, 500 μg mannan/ml, 100 μg mannan/ml, or 50 μg mannan/ml, or a range defined by any two of the preceding values.

In some embodiments of the methods, compositions and kits, the detectable exosome-binding agent comprises a lectin, an antibody, or an antibody fragment. In some embodiments, the exosome-binding agent and/or the lectin immobilized on the substrate is selected from the group consisting of Galanthus nivalis lectin (GNA), Narcissus pseudonarcissus lectin (NPA), Allium sativum lectin (ASA), Lens culinaris lectin (LCH), Sambucus nigra lectin (SNA), Maackia amurensis lectin (MAL), and concanavalin A. In some embodiments, the lectin is GNA, NPA, SNA, or MAL. In particular embodiments, the lectin is GNA. In some embodiments, the detectable exosome-binding agent comprises a label selected from the group consisting of an enzyme, a chemiluminescent agent, a fluorescent agent, and an isotope.

In some embodiments, the substrate comprises a material selected from the group consisting of sepharose, latex, glass, polystyrene, polyvinyl, nitrocellulose and silicon. In particular embodiments, the substrate comprises a multiwell plate, e.g., a 96-well plate.

In some embodiments of the methods, compositions and kits provided herein, a substrate such as a 96-well plate can be prepared with a detectable exosome-binding agent, e.g., a lectin such as GNA, by contacting the substrate with a solution of the lectin. In some embodiments, the concentration of the lectin solution is, is about, is at least, or is at least about, 0.5 μg/ml, 1.25 μg/ml, 2.5 μg/ml 5 μg/ml, 10 μg/ml, 20 μg/ml, 50 μg/ml, 100 μg/ml or a range defined by any two of the preceding values. In a preferred embodiment, the concentration of the lectin solution is 10 μg/ml.

### Diagnostics

Some embodiments of the methods and compositions provided herein include methods for the diagnosis, prognosis, or therapeutic prediction of a cancer in a subject. Some embodiments include obtaining a sample from the subject, and quantifying the exosomes present in the sample, wherein the presence and/or quantity of exosomes in the sample is indicative that the subject may have cancer.

More embodiments include methods for monitoring progression of a cancer in a subject. Some such embodiments include obtaining a first sample from a subject having cancer at a first timepoint, quantifying exosomes in the first sample, obtaining a second sample from the subject having cancer at a second timepoint, the second timepoint occurring after the first timepoint, quantifying exosomes in the second sample, wherein a change in the quantity of exosomes from the first sample to the second sample is indicative of progression of the cancer. In some embodiments, an increase in the quantity of exosomes is indicative of increased cancer.

More embodiments include monitoring therapeutic efficacy of an anti-cancer treatment. Some such embodiments include obtaining a first sample from a subject having cancer at a first timepoint, quantifying exosomes from the first sample, obtaining a second sample from the subject having cancer at a second timepoint, the second timepoint occurring after the first timepoint, quantifying exosomes from the second sample; wherein a reduction or no change in the quantity of exosomes from the second sample compared to the quantity of exosomes from the first sample indicates therapeutic efficacy of the anti-cancer treatment. Examples of anti-cancer treatment include but are not limited to surgery, radiology, chemotherapy, or a targeted cancer treatment. Targeted cancer treatment can include but are not limited to administration of small molecules, monoclonal antibodies, cancer vaccines, antisense, siRNA, aptamers, gene therapy and combinations thereof.

While the present invention has been described in some detail for purposes of clarity and understanding, one skilled in the art will appreciate that various changes in form and detail can be made without departing from the true scope of the invention.

### Examples

### Example 1—Preparing a Lectin-Coated Substrate

A lectin coated 96-well plate was prepared by adding 100 μl 10 μg/ml Galanthus nivalis lectin (GNA) to each well, and incubating for 1 hour at ambient temperature. The GNA solution was removed from the wells. The immobilized lectin was blocked with 200 μl 1% BSA+0.1% Tween 20 in 1× Dulbecco's phosphate buffered saline (dPBS) for 1 hour at ambient temperature.

### Example 2—Quantifying Mannan Bound on a Lectin-Coated Substrate

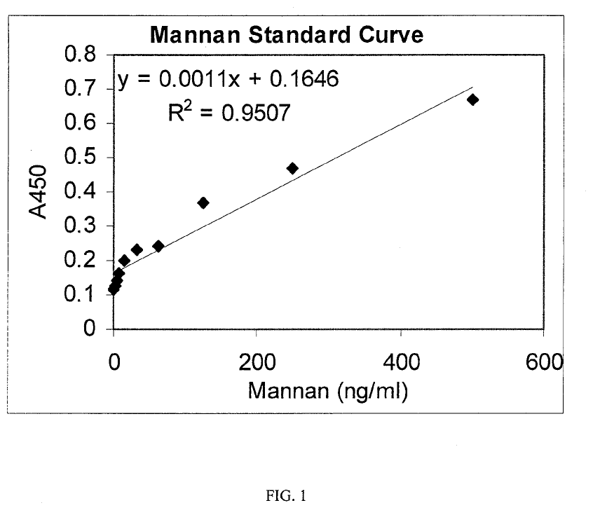

To generate a standard mannan curve, 100 μl per well of a mannan solution was incubated in each well on a plate prepared as in Example 1 for 1 hour at ambient temperature. Solutions containing 100 ng/ml, 50 ng/ml, 25 ng/ml, 12.5 ng/ml, 6.25 ng/ml, 3.125 ng/ml, and 1.5625 ng/ml of mannan in 1×dPBS, and a blank with no mannan (dPBS alone), was used to create the standard curve. Mannan solution was washed off of the plate 1× with 300 μl of 0.1% Tween 20 in dPBS (wash solution) and bound mannan was detected by incubation for 1 hour at ambient temperature with 100 μl of horse radish peroxidase (HRP) labeled GNA (1 μg/ml). The plate was washed 4× with wash solution, and detected with 100 μl tetramethylbenzadine (TMB) (SIGMA-Aldrich). TMB was incubated up to 30 minutes, or until color was seen in the blank. The TMB HRP reaction was stopped with 100 μl 1 M sulfuric acid. Resulting yellow color was measured in a plate reader at 450 nm wavelength. The resulting standard curve is shown in FIG. 1.

### Example 3—Quantifying Mannan Beads on a Lectin Substrate

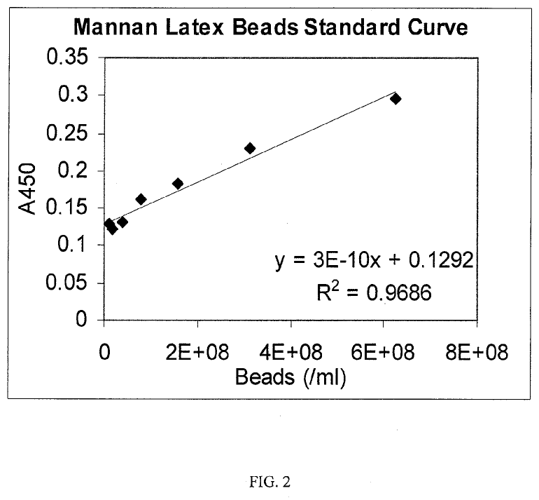

Mannan coated beads were prepared by incubating mannan at 1 mg/ml with 1e14 fluorescent latex beads (100 nm diameter), which coats the beads such that they can be captured by GNA. To prepare a standard curve, the mannan coated beads were incubated on a plate prepared as in Example 1. 100 μl of the mannan beads were incubated on the plate for 1 hour at ambient temperature at 1e10 beads/ml, 5e09 beads/ml, 2.5e09 beads/ml, 1.25e09 beads/ml, 6.25e08 beads/ml, 3.125e08 beads/ml, and 1.56e08 beads/ml, 7.81e07 beads/ml, 3.91e07 beads/ml, 1.95e07 beads/ml, 9.77e06 beads/ml in 1×dPBS, and a blank with no mannan beads (dPBS alone), to create a standard curve, part of this standard curve is shown in FIG. 2. Mannan bead solution was washed off of the plate 1× with 300 μl of 0.1% Tween 20 in dPBS (wash solution), and bound mannan beads were detected by incubation for 1 hour at ambient temperature with 100 μl of HRP labeled GNA (1 μg/ml). The plate was washed 4× with wash solution, and detected with 100 μl of TMB. TMB was incubated up to 30 minutes, or until color was seen in the blank. The TMB HRP reaction was stopped with 1 M sulfuric acid. Resulting yellow color was measured in a plate reader at 450 nm wavelength.

### Example 4—Quantifying Ovarian Cancer Cell Exosomes

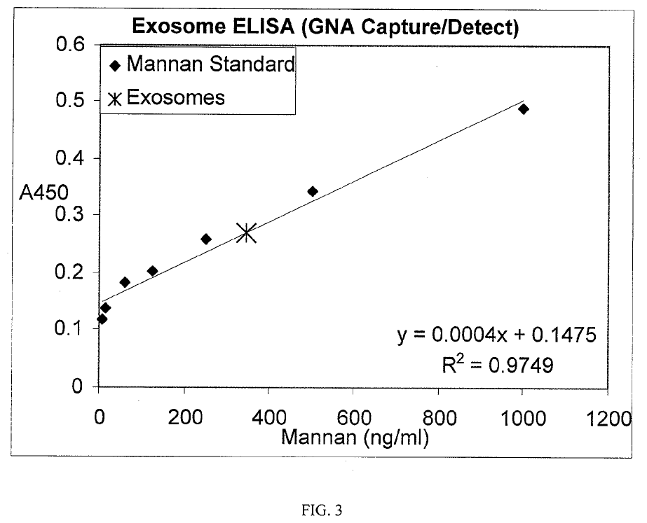

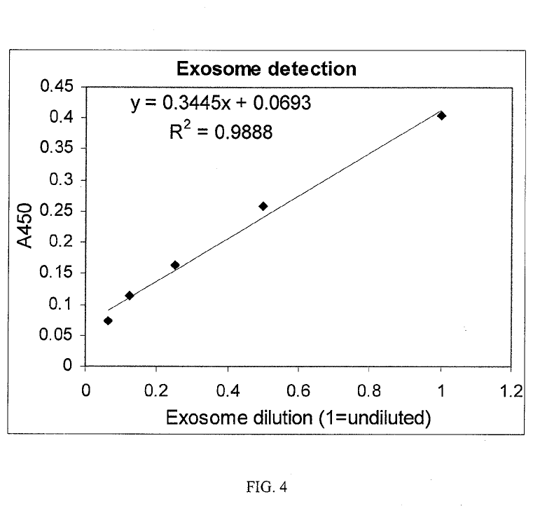

An exosome sample from ovarian cancer cells was obtained and treated according to the methods of Examples 2 and 3 using plates prepared as in Example 1. The signal generated from the ovarian cancer exosomes was correlated with the mannan and mannan bead standard curves from Examples 2 and 3; mannan coated beads are approximately the same size as the ovarian cancer exosomes. Results from such experiments are shown in FIG. 3 and FIG. 4.

### Example 5—Quantifying Tuberculosis Bacillus (TB) Infected Macrophage Exosomes

A purified exosome sample from tuberculosis bacillus (TB) infected macrophages is incubated by the same method and on the same plate as the mannan or mannan latex bead standard curves described in Example 1, 2 and 3. The signal generated from the TB infected macrophage derived exosomes is correlated with a known amount of mannan or known number of mannan coated beads that are of approximately the same size as the TB infected macrophage derived exosome.

### Example 6—Optimizing Mannan-Coated Beads

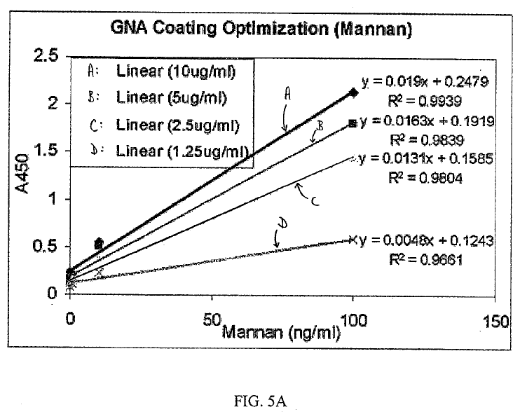

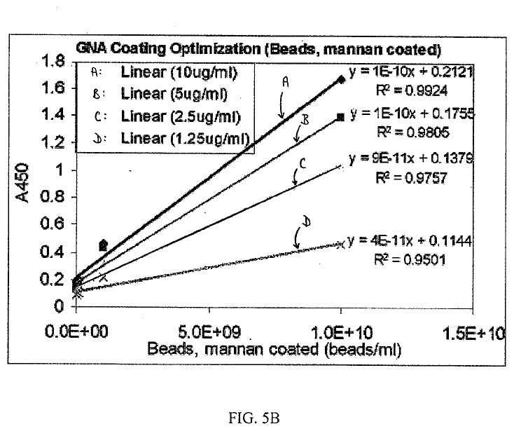

The lectin ELLSA assay was optimized with different concentrations of coated GNA. In one experiment, 100 μl of GNA in dPBS was incubated for 1 hour at ambient temperature in the wells of a 96 well plate at the following concentrations: 10 μg/ml, 5 μg/ml, 2.5 μg/ml, and 1.25 μg/ml. The GNA coating solution was then removed from the plate, and the plate blocked with 300 μl of 1% BSA, 0.1% Tween 20 for 1 hour at ambient temperature. Either mannan or mannan coated beads were incubated on the plate for 1 hour at ambient temperature at 1000 ng/ml, 100 ng/ml, 10 ng/ml and 1 ng/ml for mannan and Tell beads/ml, 1e10 beads/ml, 1e9 beads/ml, and 1e8 beads/ml for mannan latex beads, including 2 blank wells (dPBS alone) per GNA coating concentration to determine sensitivity and background of the assay. Mannan or mannan beads were incubated for 1 hour at ambient temperature on the GNA coated plates. Wells were washed 1× with 300 μl wash solution. 100 μl of HRP labeled GNA (1 μg/ml) was incubated with bound mannan or mannan beads for 1 hour at ambient temperature. Wells were washed 4× with 300 μl wash solution, and 100 μl of TMB was added to each well for 30 minutes or until color developed in the blank wells. The TMB HRP reaction was stopped with 1 M sulfuric acid, and the resulting yellow color was measured in a plate reader at 450 nm wavelength. The results are shown in FIGS. 5A and 5B. Substrates coated with 10 μg/ml GNA provided optimal results in this experiment.

### Example 7—Quantifying Colon Cancer Exosomes

A 96-well plate was prepared according to Example 1. Various dilutions of cell culture media supernatants from human primary colon cancer cells were obtained.

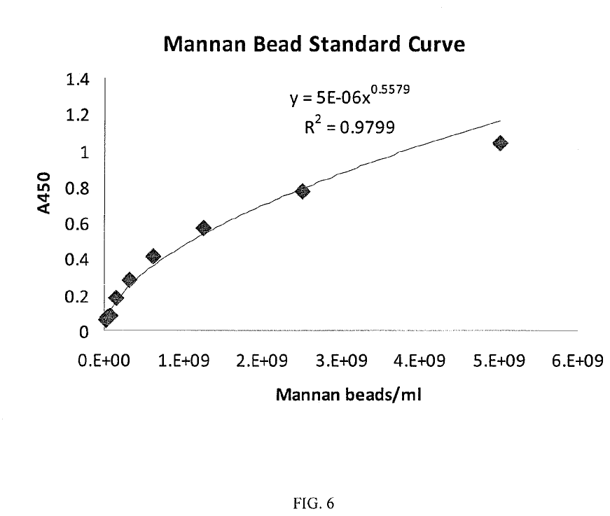

A 100 μl sample of a mannan bead standard or a supernatant sample was added to each well, and incubated for 1 hour at RT. The plates were washed 1× with PBS+0.1% Tween-20. Bound exosomes or bound mannan was detected by adding 100 μl 1 μg/ml GNA coupled to horse radish peroxidase (HRP), and incubating for 1 hour at RT, washing the plate 4× with PBS+0.1% Tween-20. HRP was detected and measured by adding 100 μl of tetramethylbenzidine, allowing the color to develop 30 minutes at RT, stopping the reaction with 100 μl 1 M sulfuric acid. Intensity of color was measured at an absorbance at 450 nm. FIG. 6 shows the mannan bead standard curve. Table 1 summarizes the calculated exosome concentration relative to the mannan standard curve.

TABLE 1

| | | Exosome concentration relative |
|---|---|---|
| Supernatant | | to mannan bead standard curve |
| dilution | A₄₅₀ | (Beads/ml) |
| 1.00 | 1.019 | 3.28E+09 |
| 0.1 | 0.08 | 3.43E+07 |
| 0.01 | 0.039 | 9.47E+06 |
| 0.001 | 0.028 | 5.23E+06 |

### Example 8—Quantifying Melanoma Exosomes

Melanoma exosome cell culture supernatants were prepared by plating out five melanoma cell lines (human primary melanoma cell lines). After five days, the cells were washed and then incubated for 48 hours in supplemented media. The supernatants were collected.

Exosomes were detected and measured as described in Example 7. Table 2 summarizes the Absorbance 450 nm measurements of diluted supernatants and the concentration of the exosomes relative to a mannan standard curve.

TABLE 2

| | | | Exosome | |
|---|---|---|---|---|
| | | | concentration | Average |
| | | | relative to | relative |
| | | | mannan | exosome |
| | Supernatant | | standard curve | concentration |
| Cell line | dilution | A₄₅₀ | (ng/ml) | (ng/ml) |
| CCS-1 | Undiluted | 0.298 | 24 | 25 |
| | 1:2 | 0.211 | 26 | |
| | 1:4 | 0.152 | 25 | |
| CCS-2 | Undiluted | 0.531 | 51 | 45 |
| | 1:2 | 0.267 | 40 | |
| | 1:4 | 0.191 | 43 | |
| CCS-3 | Undiluted | 0.171 | 8 | 17 |
| | 1:2 | 0.187 | 21 | |
| | 1:4 | 0.144 | 21 | |
| CCS-4 | Undiluted | 0.388 | 34 | 45 |
| | 1:2 | 0.313 | 51 | |
| | 1:4 | 0.203 | 49 | |
| CCS-5 | Undiluted | 0.263 | 19 | 20 |
| | 1:2 | 0.182 | 20 | |
| | 1:4 | 0.147 | 22 | |

### Example 9—Quantifying Ovarian Cancer Exosomes

Various dilutions of human ascites fluid from a ovarian cancer patient were obtained. Exosomes in the fluid were quantified according to a method similar to the method described in Example 7. Table 3 summarizes the calculated concentrations of the ovarian exosomes relative to mannan.

TABLE 3

| | | Exosome concentration relative |
|---|---|---|
| Supernatant | | to mannan standard curve |
| dilution | A₄₅₀ | (ng/ml) |
| 1 | 0.404 | 55.6 |
| 0.5 | 0.259 | 35.4 |
| 0.25 | 0.163 | 22.1 |
| 0.125 | 0.115 | 15.4 |
| 0.0625 | 0.073 | 9.6 |
| 0 | 0 | −0.5 |

### Example 10—Quantifying Exosomes Derived from Tuberculosis Infected Macrophage

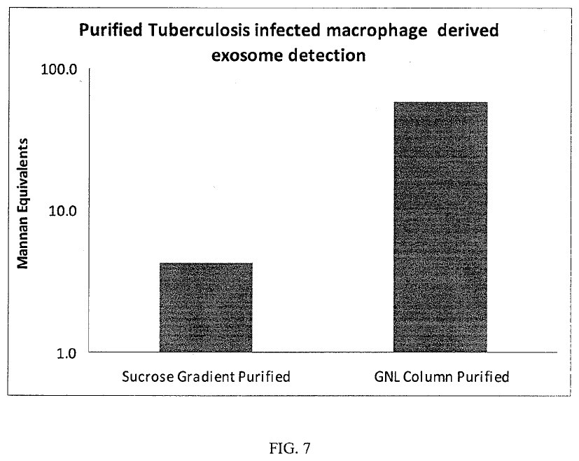

Exosomes from murine tuberculosis infected macrophage were purified by using a sucrose gradient, or using a GNA resin and eluting with 1 M alpha methyl-mannoside. Exosome concentrations were determined using a method similar to the method of Example 7. FIG. 7 summarizes the results.

### Example 11—Quantifying Ovarian Cancer Exosomes with Anti-PLAP Antibody

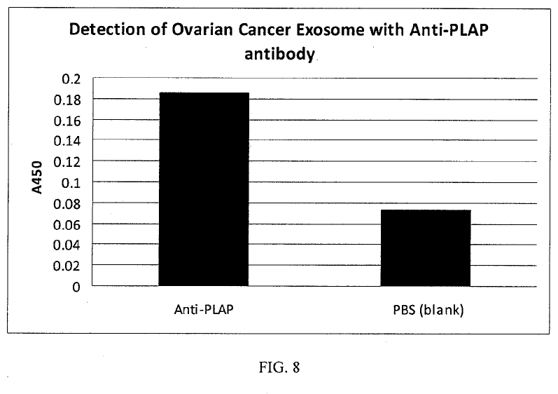

The ability of anti-placental alkaline phosphatase (PLAP) to detect ovarian cancer exosomes bound to GNA coated plates was tested. GNA coated multiwell plates were prepared as described in Example 1. Ovarian cancer cell exosomes were incubated in the wells. The presence of exosomes was determined using labeled anti-PLAP antibody which detects a marker of ovarian cells. The results are summarized in FIG. 8.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

1. (canceled)

2. A method for characterizing exosomes in a biological sample comprising:
(a) increasing the concentration of exosomes in the sample to generate a concentrated exosome sample;
(b) determining a quantity of lectin bound exosomes in a first portion of the concentrated exosome sample, comprising:
contacting the first portion of the concentrated exosome sample with a lectin immobilized on a multi-well plate;
contacting exosomes bound to the immobilized lectin with a detectable lectin;
measuring a signal from the bound detectable lectin;
measuring a control signal of known quantities of a lectin-binding compound to establish a standard curve; and
comparing the signal from the bound detectable lectin to the standard curve;
(c) determining a quantity of antibody bound exosomes in a second portion of the concentrated exosome sample, comprising:
contacting a second portion of the concentrated exosome sample with an antibody immobilized on a multi-well plate;
contacting exosomes bound to the immobilized antibody with a detection antibody;
measuring a signal from the bound detection antibody;
measuring a control signal from known quantities of an antibody-binding compound to establish a standard curve;
comparing the signal from the bound antibody to the standard curve; and
(d) comparing the quantity of lectin bound exosomes in the first portion of the concentrated exosome sample to the quantity of antibody bound exosomes in the second portion of the concentrated exosome sample.

3. The biological sample of claim 2, wherein the sample comprises heterogenous exosomes.

4. The method of claim 2, wherein increasing the concentration of exosomes in the sample comprises subjecting the sample to a method selected from the group consisting of size exclusion chromatography, density gradient centrifugation, differential centrifugation, nanomembrane ultrafiltration, immunoabsorbent capture, affinity purification, and microfluidic separation, or a combination thereof.

5. The method of claim 2, wherein the biological sample is selected from the group consisting of peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, umbilical cord blood, and ascites fluid.

6. The method of claim 2, wherein the lectin immobilized on the multiwell plate is selected from the group consisting of Galanthus nivalis lectin (GNA) Narcissus pseudonarcissus lectin (NPA), Allium sativum lectin (ASA), Lens culinaris lectin (LCH), Sambucus nigra lectin (SNA), Maackia amurensis lectin (MAL), and concanavalin A.

7. The method of claim 2, wherein the antibody bound exosomes in the second portion of the concentrated exosome sample are comprised of cancer exosomes.

8. The method of claim 6, wherein the cancer is breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC), or gastric cancer.

9. The method of claim 2, wherein the method provides a concentrated exosome sample that is at least about 1×10⁹ exosomes/ml.

10. The method of claim 2, wherein the sample comprises an exosome having a diameter of about 10 nm to about 800 nm.

11. The method of claim 2, wherein the detectable lectin comprises a detectable label selected from the group consisting of an enzyme, a chemiluminescent agent, a fluorescent agent, and an isotope.

12. The method of claim 2, wherein the multiwell plate comprises a material selected from the group consisting of sepharose, latex, glass, polystyrene, polyvinyl and silicon.

13. The method of claim 2, wherein the biological sample is mammalian.

14. The method of claim 2, wherein the antibody immobilized on the multiwell plate detects a cancer marker.

15. The method of claim 2, wherein the antibody immobilized on the multiwell plate detects ovarian cancer, colon cancer or melanoma.

16. The method of claim 2, wherein the antibody immobilized on the multiwell plate is an anti-placental alkaline phosphatase (PLAP) antibody.

17. The method of claim 2, wherein the lectin immobilized on a substrate and the detectable lectin are capable of binding to exosomes derived from a plurality of cell types.
